(19) United States
(12) Patent Application Publication
(10) Pub. No.: US 2026/0144555 A1
(43) Pub. Date: May 28, 2026

(54) THROMBUS REMOVAL SYSTEMS AND ASSOCIATED METHODS

(71) Applicant: Shifamed Holdings, LLC, Campbell, CA (US)

(72) Inventors: Paul GUNNING, Montara, CA (US); Tom SAUL, Portland, OR (US); Nicholas LYFORD, Saratoga, CA (US)

(21) Appl. No.: 19/123,712

(22) PCT Filed: Oct. 26, 2023
(86) PCT No.: PCT/US2023/077912

§ 371 Date: Apr. 23, 2025

Related U.S. Application Data
(60) Provisional application No. 63/381,019, filed on Oct. 26, 2022.
(60) Provisional application No. 63/381,725, filed on Oct. 31, 2022.

(51) Int. Cl. A61B 17/22; A61B 17/00

(52) U.S. Cl. CPC A61B 17/22; A61B 17/00234; A61B 2017/00238; A61B 2017/00305; A61B 2017/00477; A61B 2017/00544; A61B 2017/00561; A61B 2017/22079; A61B 2217/005

(57) ABSTRACT

The present technology relates to systems and methods for removing a thrombus from a blood vessel of a patient. In some embodiments, the present technology is directed to systems including an elongated catheter having a distal portion configured to be positioned within the blood vessel of the patient, a proximal portion configured to be external to the patient, and a lumen extending therebetween. The system can also include a fluid delivery mechanism coupled with a fluid lumen and configured to apply fluid to at least partially fragment the thrombus.

### PRIORITY CLAIM

This patent application claims priority to U.S. provisional patent application No. 63/381,019, titled “THROMBUS REMOVAL SYSTEMS AND ASSOCIATED METHODS,” and filed on Oct. 26, 2022, and U.S. provisional patent application No. 63/381,725, titled “THROMBECTOMY SYSTEM AND METHOD OF REMOVING THROMBUS,” and filed on Oct. 31, 2022, both of which are herein incorporated by reference in their entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present technology generally relates to medical devices and, in particular, to systems including aspiration and fluid delivery mechanisms and associated methods for removing a thrombus from a mammalian blood vessel.

### BACKGROUND

Thrombotic material may lead to a blockage in fluid flow within the vasculature of a mammal. Such blockages may occur in varied regions within the body, such as within the pulmonary system, peripheral vasculature, deep vasculature, or brain. Pulmonary embolisms typically arise when a thrombus originating from another part of the body (e.g., a vein in the pelvis or leg) becomes dislodged and travels to the lungs. Anticoagulation therapy is the current standard of care for treating pulmonary embolisms, but may not be effective in some patients. Additionally, conventional devices for removing thrombotic material may not be capable of navigating the tortuous vascular anatomy, may not be effective in removing thrombotic material, and/or may lack the ability to provide sensor data or other feedback to the clinician during the thrombectomy procedure. Existing thrombectomy devices operate based on simple aspiration which works sufficiently for certain clots but is largely ineffective for difficult, organized clots. Many patients presenting with deep vein thrombus (DVT) are left untreated as long as the risk of limb ischemia is low. In more urgent cases, they are treated with catheter-directed thrombolysis or lytic therapy to break up a clot over the course of many hours or days. More recently other tools like clot retrievers have been developed to treat DVT and pulmonary embolism (PE), but these tools are not being widely adopted because of their limited effectiveness and additional costs versus aspiration or the standard of case. Other recent developments focus on slicing or macerating the clot, but these mechanisms are designed to reduce the risk of the catheter clogging and do not address the problem of tough, large, organized clots. There remains the need for a device to address these and other problems with existing venous thrombectomy including, but not limited to, a fast, easy-to-use, and effective device for removing a variety of clot morphologies.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1-1L illustrate various views of a portion of a thrombus removal system including a distal portion of an elongated catheter configured in accordance with an embodiment of the present technology.

FIGS. 2A-2E illustrate plan views of various configurations of irrigation ports and fluid streams of a thrombus removal system according to embodiments of the present technology.

FIGS. 3A-3H illustrate an elevation view of various configurations of irrigation ports and fluid streams of a thrombus removal system according to embodiments of the present technology.

FIGS. 4A-4C illustrate various embodiments of a thrombus removal system including a saline source, an aspiration system, and one or more controls for controlling irrigation and/or aspiration of the system.

FIGS. 4D-4E illustrate an introducer system for introducing a medical device into the vasculature of a subject.

FIGS. 5A-5B illustrate one embodiment of a funnel of a thrombus removal system including an adjustment mechanism for changing a shape and/or size of the funnel.

FIG. 6 shows an introducer catheter of a thrombus removal system.

FIGS. 7A-7F illustrate operation of an adjustment mechanism and introducer catheter for changing a shape and/or size of the funnel.

FIGS. 8A-8D show side profiles of a funnel with an adjustment mechanism for changing a shape and/or size of the funnel.

FIGS. 9A-9D show top-down views of a funnel with an adjustment mechanism for changing a shape and/or size of the funnel.

FIGS. 10A-10C show top-down views of a funnel with an adjustment mechanism for changing a shape and/or size of the funnel.

FIGS. 11A-11B illustrate another embodiment of a funnel with an adjustment mechanism for changing a shape and/or size of the funnel.

### SUMMARY OF THE DISCLOSURE

A thrombus removal is provided, comprising an elongate shaft comprising a working end, at least one fluid lumen in the elongate shaft, and two or more apertures disposed at or near the working end, the two or more apertures in fluid communication with the least one fluid lumen and configured to generate two or more fluid streams to mechanically fractionate a target thrombus.

A thrombus removal device, comprising: an elongate shaft; at least one aspiration lumen in the elongate shaft; a funnel disposed at or near a distal end of the elongate shaft; and an adjustable frame attached to the funnel and axially adjustable relative to the elongate shaft, wherein axial movement of the adjustable frame is configured to adjust a diameter of the funnel and/or a shape of the funnel.

In some aspects, axial movement of the adjustable frame is configured to reduce a diameter of the funnel.

In some aspects, axial movement of the adjustable frame is configured to reduce a diameter of a base portion of the funnel.

In other aspects, axial movement of the adjustable frame is configured to reduce a diameter of a central portion of the funnel.

In some aspects, axial movement of the adjustable frame is configured to reduce a diameter of a distal portion of the funnel.

In some aspects, axial movement of the adjustable frame is configured to increase a diameter of the funnel.

In other aspects, axial movement of the adjustable frame is configured to increase a diameter of a base portion of the funnel.

In some aspects, axial movement of the adjustable frame is configured to increase a diameter of a central portion of the funnel.

In some aspects, axial movement of the adjustable frame is configured to increase a diameter of a distal portion of the funnel.

In one aspect, the adjustable frame comprises: a support frame collar assembly disposed around the elongate shaft and configured to move axially relative to the elongate shaft; and one or more frame ribs coupled to the support frame collar assembly and to the funnel.

In one aspect, the device includes a gap between the support frame collar and the elongate shaft.

In some aspects, the device includes a catheter sheath disposed over the elongate shaft, wherein the adjustable frame comprises one or more engagement features configured to couple with one or more engagement features of the catheter sheath.

In one aspect, axial movement of the catheter sheath relative to the elongate shaft is configured to cause a corresponding axial movement of the adjustment frame when the catheter shaft is coupled to the adjustment frame.

In another aspect, axial movement of the adjustable frame is configured to cause the funnel to assume an oval shape.

In some aspects, axial movement of the adjustable frame is configured to cause the funnel to assume a skived shape.

In additional aspects, axial movement of the adjustable frame is configured to cause the funnel to assume a clover shape.

In some aspects, the funnel comprises a circular cross section shape when at-rest, and wherein axial movement of the adjustable frame causes the funnel to assume a non-circular cross-section shape.

In other aspects, the funnel comprises a compliant material and does not include a separate funnel frame for structure.

In some aspects, the device includes one or more coupling structures attached to the adjustable frame and extending proximally towards a handle of the device.

In one aspect, the one or more coupling structures comprise pull wires.

In other aspects, manipulation of the one or more coupling structures causes axial movement of the adjustable frame

A method is provided, comprising; introducing a funnel of a thrombus removal device to a thrombus location in a blood vessel; and actuating a frame of the funnel to change a shape and/or diameter of the funnel.

In some aspects, the frame is external to the funnel.

In some aspects, the frame is axially adjustable relative to an elongate shaft of the thrombus removal device.

In one aspect, the method includes engaging an introducer sheath with the frame, wherein actuating the frame further comprises actuating the frame with the introducer sheath.

In some aspects, engaging the introducer sheath with the frame comprises rotating the introducer sheath to engage an engagement feature of the introducer sheath with a corresponding engagement feature of the frame.

In some aspects, actuating the frame comprises adjusting an axial position of the frame relative to a shaft of the thrombus removal device with the introducer sheath.

In one aspect, actuating the frame comprises actuating the frame to reduce a diameter of the funnel.

In some aspects, actuating the frame comprises actuating the frame to reduce a diameter of a base portion of the funnel.

In some aspects, actuating the frame comprises actuating the frame to reduce a diameter of a central portion of the funnel.

In some aspects, actuating the frame comprises actuating the frame to reduce a diameter of a distal portion of the funnel.

In another aspect, actuating the frame comprises actuating the frame to increase a diameter of the funnel.

In one aspect, actuating the frame comprises actuating the frame to increase a diameter of a base portion of the funnel.

In another aspect, actuating the frame comprises actuating the frame to increase a diameter of a central portion of the funnel.

In some aspects, actuating the frame comprises actuating the frame to increase a diameter of a distal portion of the funnel.

In another aspect, actuating the frame comprises changing a shape of the funnel from a generally circular cross section at-rest shape to a non-circular cross-section shape.

A method is provided, comprising; introducing an elongate shaft and a funnel of a thrombus removal device to a thrombus location in a blood vessel; and moving a frame connected to the funnel in an axial direction with respect to the elongate shaft to change a shape and/or diameter of the funnel.

In some aspects, changing the shape of the funnel comprises changing a cross-sectional shape of the funnel from generally circular to non-circular.

In one aspect, the frame is external to the funnel.

In another aspect, the frame is internal to the funnel.

In some aspects, the method includes engaging an introducer sheath with the frame, wherein actuating the frame further comprises actuating the frame with the introducer sheath.

In some aspects, engaging the introducer sheath with the frame comprises rotating the introducer sheath to engage an engagement feature of the introducer sheath with a corresponding engagement feature of the frame.

In some aspects, actuating the frame comprises adjusting an axial position of the frame relative to a shaft of the thrombus removal device with the introducer sheath.

In some aspects, actuating the frame comprises actuating the frame to reduce a diameter of the funnel.

In other aspects, actuating the frame comprises actuating the frame to reduce a diameter of a base portion of the funnel.

In some aspects, actuating the frame comprises actuating the frame to reduce a diameter of a central portion of the funnel.

In another aspect, actuating the frame comprises actuating the frame to reduce a diameter of a distal portion of the funnel.

In some aspects, actuating the frame comprises actuating the frame to increase a diameter of the funnel.

In another aspect, actuating the frame comprises actuating the frame to increase a diameter of a base portion of the funnel.

In some aspects, actuating the frame comprises actuating the frame to increase a diameter of a central portion of the funnel.

In some aspects, actuating the frame comprises actuating the frame to increase a diameter of a distal portion of the funnel.

In some aspects, actuating the frame comprises changing a shape of the funnel from a generally circular cross section at-rest shape to a non-circular cross-section shape.

In another aspect, the method further comprises locking an axial position of the introducer sheath relative to the frame.

In some aspects, locking the axial position further comprises locking the introducer sheath in an introducer hub.

In some aspects, the introducer sheath comprises a Touhy Borst.

### DETAILED DESCRIPTION

This application is related to disclosure in International Application No. PCT/US2021/020915, filed Mar. 4, 2021 (the '915 application), and International Application No. PCT/US2022/033024, filed Jun. 10, 2022 (the '024 application), the disclosures of which are incorporated by reference herein for all purposes. The '915 and '024 applications describe general mechanisms for capturing and removing a clot. By example, multiple fluid streams are directed toward the clot to fragment the material.

The present technology is generally directed to thrombus removal systems and associated methods. A system configured in accordance with an embodiment of the present technology can include, for example, an elongated catheter having a distal portion configured to be positioned within a blood vessel of the patient, a proximal portion configured to be external to the patient, a fluid delivery mechanism configured to fragment the thrombus with pressurized fluid, an aspiration mechanism configured to aspirate the fragments of the thrombus, and one or more lumens extending at least partially from the proximal portion to the distal portion.

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to the figures.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

Although some embodiments herein are described in terms of thrombus removal, it will be appreciated that the present technology can be used and/or modified to remove other types of emboli that may occlude a blood vessel, such as fat, tissue, or a foreign substance. Additionally, although some embodiments herein are described in the context of thrombus removal from a pulmonary artery (e.g., pulmonary embolectomy), the technology may be applied to removal of thrombi and/or emboli from other portions of the vasculature (e.g., in neurovascular, coronary, or peripheral applications). Moreover, although some embodiments are discussed in terms of maceration of a thrombus with a fluid, the present technology can be adapted for use with other techniques for breaking up a thrombus into smaller fragments or particles (e.g., ultrasonic, mechanical, enzymatic, etc.).

The headings provided herein are for convenience only and do not interpret the scope or meaning of the claimed present technology.

### Systems for Thrombus Removal

As provided above, the present technology is generally directed to thrombus removal systems. Such systems include an elongated catheter having a distal portion positionable within a blood vessel of the patient (e.g., an artery or vein), a proximal portion positionable outside the patient's body, a fluid delivery mechanism configured to fragment the thrombus with pressurized fluid, an aspiration mechanism configured to aspirate the fragments of the thrombus, and one or more lumens extending at least partially from the proximal portion to the distal portion. In some embodiments, the systems herein are configured to engage a thrombus in a patient's blood vessel, break the thrombus into small fragments, and aspirate the fragments out of the patient's body. The pressurized fluid streams (e.g., jets) function to cut or macerate thrombus, before, during, and/or after at least a portion of the thrombus has entered the aspiration lumen or a funnel of the system. Fragmentation helps to prevent clogging of the aspiration lumen and allows the thrombus removal system to macerate large, firm clots that otherwise could not be aspirated. As used herein, “thrombus” and “embolism” are used somewhat interchangeably in various respects. It should be appreciated that while the description may refer to removal of “thrombus,” this should be understood to encompass removal of thrombus fragments and other emboli as provided herein.

According to embodiments of the present technology, a fluid delivery mechanism can provide a plurality of fluid streams (e.g., jets) to fluid apertures of the thrombus removal system for macerating, cutting, fragmenting, pulverizing and/or urging thrombus to be removed from a proximal portion of the thrombus removal system. The thrombus removal system can include an aspiration lumen extending at least partially from the proximal portion to the distal portion of the thrombus removal system that is adapted for fluid communication with an aspiration pump (e.g., vacuum source). In operation, the aspiration pump may generate a volume of lower pressure within the aspiration lumen near the proximal portion of the thrombus removal system, urging aspiration of thrombus from the distal portion.

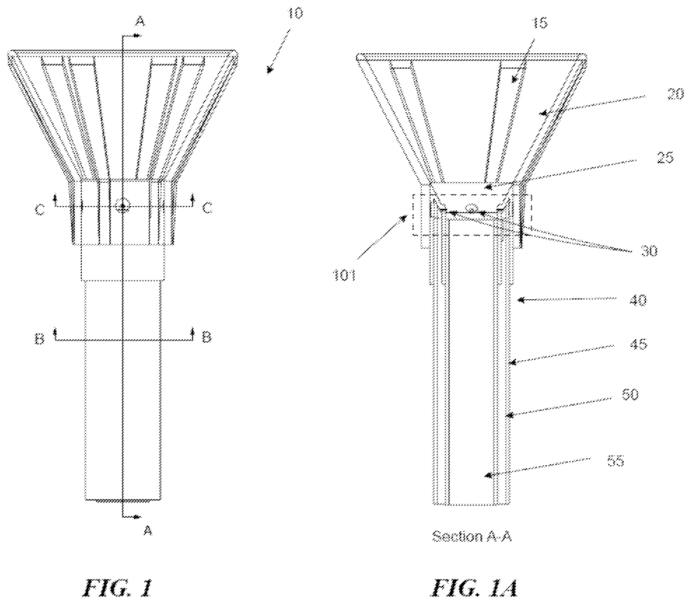

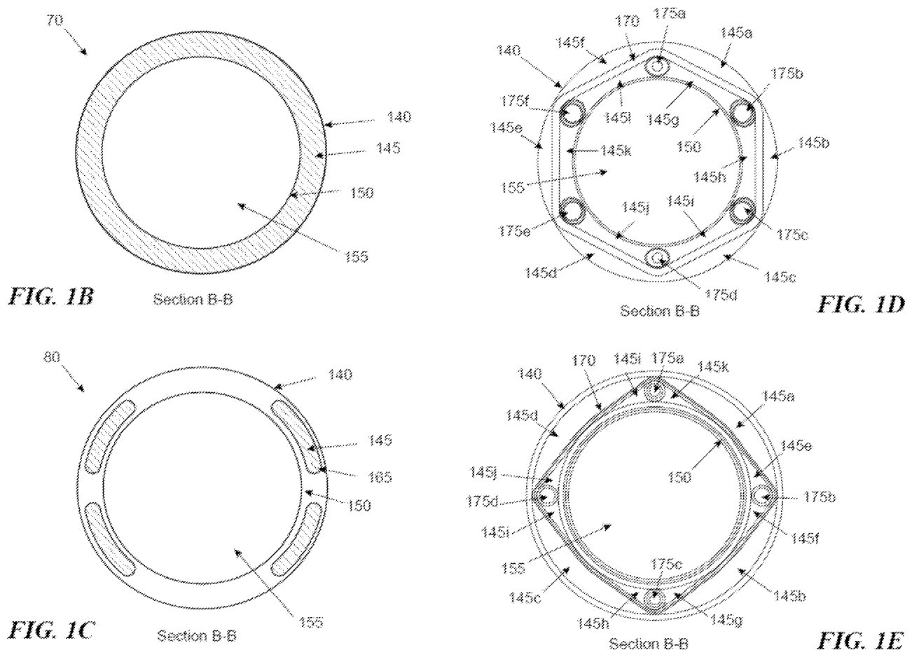

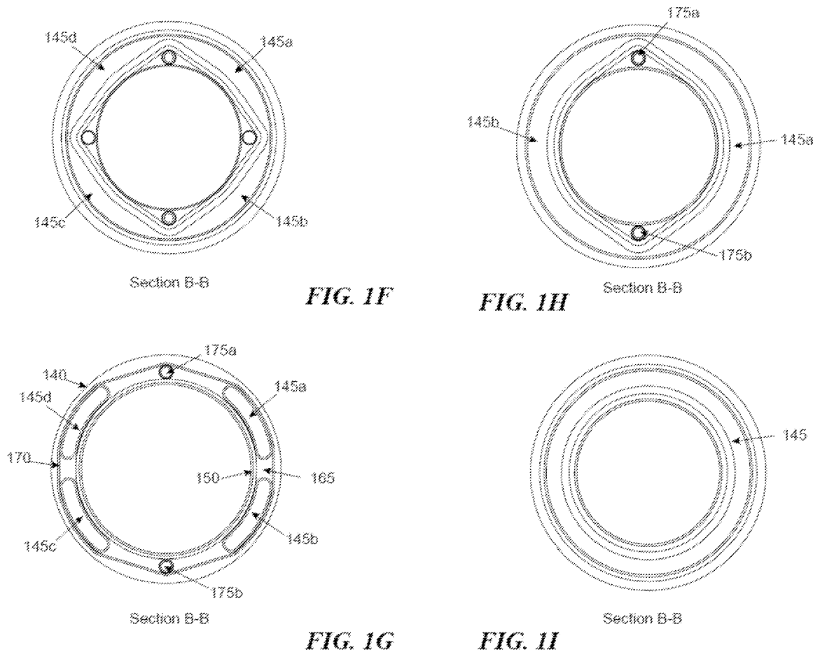

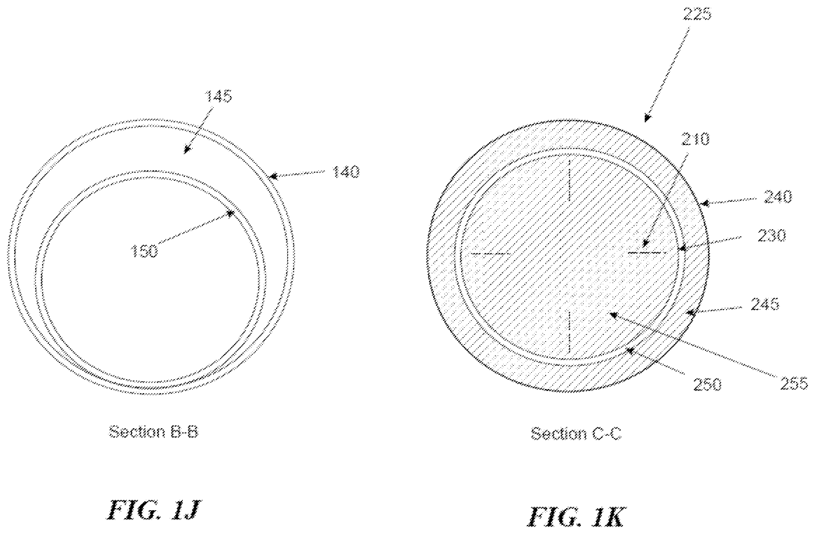

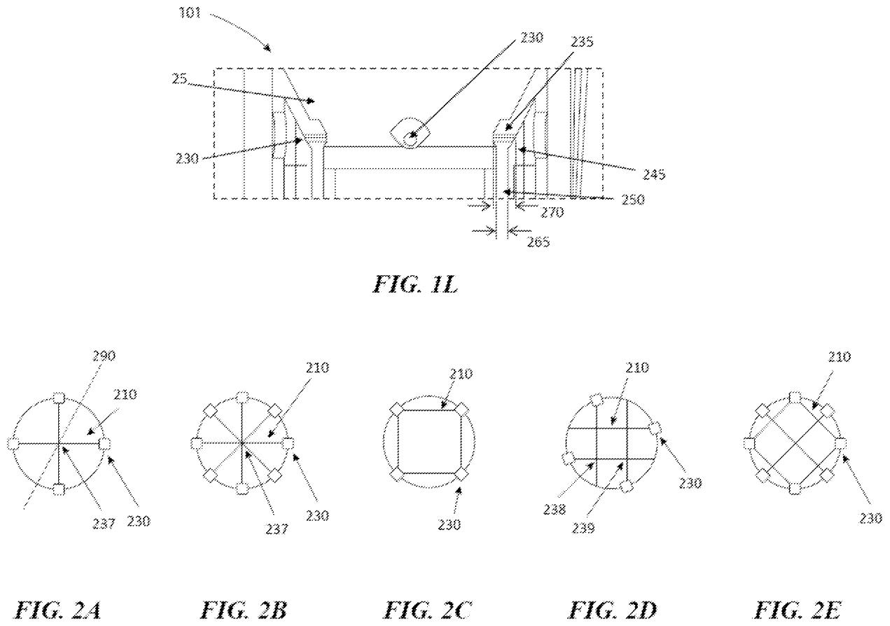

FIG. 1 illustrates a distal portion 10 of a thrombus removal system according to an embodiment of the present technology. FIG. 1A Section A-A illustrates an elevation sectional view of the distal portion. The example section A-A in FIG. 1A depicts a funnel 20 that is positioned at the distal end of the distal portion 10, the funnel adapted to engage with thrombus and/or a tissue (e.g., vessel) wall to aid in thrombus fragmentation and/or removal. The funnel can have a variety of shapes and constructions as would be understood by one of skill from the description herein. The example section A-A in FIG. 1A depicts a double walled thrombus removal device construction having an outer wall/tube 40 and an inner wall/tube 50. An aspiration lumen 55 is formed by the inner wall 50 and is centrally located. A generally annular volume forms at least one fluid lumen 45 between the outer wall 40 and the inner wall 50. The fluid lumen 45 is adapted for fluid communication with the fluid delivery mechanism. One or more apertures (e.g., nozzles, orifices, or ports) 30 are positioned in the thrombus removal system to be in fluid communication with the fluid lumen 45 and an irrigation manifold 25. In operation, the ports 30 are adapted to direct (e.g., pressurized) fluid toward thrombus that is engaged with the distal portion 10 of the thrombus removal system.

In various embodiments, the system can have an average flow velocity within the fluid lumen of up to 20 m/s to achieve consistent and successful aspiration of clots. In some embodiments, the fluid source itself can be delivered in a pulsed sequence or a preprogrammed sequence that includes some combination of pulsatile flow and constant flow to deliver fluid to the jets. In these embodiments, while the average pulsed fluid velocity may be up to 20 m/s, the peak fluid velocity in the lumen may be up to 30 m/s or more during the pulsing of the fluid source. In some embodiments, the jets or apertures are no smaller than 0.0100″ or even as small as 0.008″ to avoid undesirable spraying of fluid. In some embodiments, the system can have a minimum vacuum or aspiration pressure of 15 inHg, to remove target clots after they have been macerated or broken up with the jets described above.

The thrombus removal system can be sized and configured to access and remove thrombi in various locations or vessels within a patient's body. It should be understood that while the dimensions of the system may vary depending on the target location, generally similar features and components described herein may be implemented in the thrombus removal system regardless of the application. For example, a thrombus removal system configured to remove pulmonary embolism (PE) from a patient may have an outer wall/tube with a size of approximately 11-13 Fr, or preferably 12 Fr, and an inner wall/tube with a size of 7-9 Fr, or preferably 8 Fr. A deep vein thrombosis (DVT) device, on the other hand, may have an outer wall/tube with a size of approximately 9-11 Fr, or preferably 10 Fr, and an inner wall/tube with a size of 6-9 Fr, or preferably 7.5 Fr. Applications are further provided for ischemic stroke and peripheral embolism applications.

Section B-B of FIG. 1B illustrates in plan view a portion of the thrombus removal system that is proximal to the funnel and irrigation manifold. Section B-B depicts an outer wall 140, an inner wall 150, an aspiration lumen 155 and a fluid lumen 145. In some embodiments, in cross-section the aspiration lumen 155 is generally circular and the fluid lumen 145 is generally annular in shape (e.g., cross-section 70). It will be appreciated that alternative constructions and/or arrangements of the inner wall 150 and the outer wall 140 produce variations in cross-sectional shape of the aspiration and fluid lumens 155 and 145. For example, the inner wall 150 can be shaped to form an aspiration lumen 155 that, in cross-section, is generally oval, circular, rectilinear, square, pentagonal, or hexagonal. The inner and outer walls 150 and 140 can be shaped and arranged to form a fluid lumen 145 that, in cross-section, is generally crescent-shaped, diamond shaped, or irregularly shaped. For example, referring to FIG. 1C Section B-B, the region between the inner wall 150 and the outer wall 140 can include one or more wall structures 165 that form respective fluid lumens 145 (e.g., as in cross-section 80). The wall structures 165 can be formed by lamination between the outer and inner walls 140 and 150, or by a multi-lumen extrusion that forms a plurality of the wall structures.

Section B-B of FIGS. 1D-1H illustrate additional examples of a portion of the thrombus removal system that is proximal to the funnel and irrigation manifold. Similar to the embodiments described above, the portion in these examples can include an outer wall 140, an inner wall 150, and an aspiration lumen 155. Additionally, the illustrated portion of the thrombus removal system can include a middle wall 170 disposed between the outer wall 140 and the inner wall 150. The middle wall 170 enables further segmentation of the annular space between the inner wall and outer wall into a plurality of distinct fluid lumens and/or auxiliary lumens. For example, referring to FIG. 1D, the middle wall can be generally hexagon shaped, and the annular space can include a plurality of fluid lumens 145a-141 and a plurality of auxiliary lumens 175a-175f. As shown in FIG. 1D, the fluid lumens can be formed by some combination of the outer wall 140 and the middle wall 170, or between the middle wall 170, the inner wall 150, and two of the auxiliary lumens. For example, fluid lumen 145a is formed in the space between outer wall 140 and middle wall 170. However, fluid lumen 145g is formed in the space between middle wall 170, inner wall 150, auxiliary lumen 175a, and auxiliary lumen 175b. Generally, the fluid lumens are configured to carry a flow of fluid such as saline from a saline source of the system to one or more ports/apertures/orifices of the system. The auxiliary lumens can be configured for a number of functions. In some embodiments, the auxiliary lumens can be coupled to the fluid/saline source and to the apertures to be used as additional fluid lumens. In other embodiments, the auxiliary lumens can be configured as steering ports and can include a guide wire or steering wire within the lumen for steering of the thrombus removal system. Additionally, in other embodiments, the auxiliary lumens can be configured to carry electrical, mechanical, or fluid connections to one or more sensors. For example, the system may include one or more electrical, optical, or fluid based sensors disposed along any length of the system. The sensors can be used during therapy to provide feedback for the system (e.g., sensors can be used to detect clogs to initiate a clog removal protocol, or to determine the proper therapy mode based on sensor feedback such as jet pulse sequences, aspiration sequences, etc.). The auxiliary ports can therefore be used to connect to the sensors, e.g., by electrical connection, optical connection, mechanical/wire connection, and/or fluid connection. It is also contemplated that the fluid and auxiliary lumens can be configured to carry and deliver other fluids, such as thrombolytics or radio-opaque contrast injections to the target tissue site during treatment.

It should be understood that in some embodiments, all the fluid lumens are fluidly connected to all of the jets or apertures of the thrombus removal device. Therefore, when a flow of fluid is delivered from the fluid lumen(s) to the jets, all jets are activated with a jet of fluid at once. However, it should also be understood that in some embodiments, the fluid lumens are separate or distinct, and these distinct fluid lumens may be fluidly coupled to one or more jets but not to all jets of the device. In these embodiments, a subset of the jets can be controlled by delivering fluid only to the fluid lumens that are coupled to that subset of jets. This enables additional functionality in the device, in which specific jets can be activated in a user defined or predetermined order.

In various embodiments, the fluid pressure is generated at the pump (in the console or handle). The fluid is accelerated as it exits the ports at the distal end and is directed to the target clot. In this way a wider variety of cost-effective components can be used to form the catheter while still maintaining a highly-effective device for clot removal. Additional details are provided below.

Section B-B of FIG. 1E illustrates another embodiment of the portion of the thrombus removal system that is proximal to the funnel and irrigation manifold. Similar to the embodiment of FIG. 1D, this embodiment also includes a middle wall 170. However, the middle wall in this example is generally square shaped, facilitating the formation of fluid lumens 145a-145k and auxiliary lumens 175a-175d. The example illustrated in section B-B of FIG. 1F is similar to that of the embodiment of FIG. 1E, however this embodiment includes only fluid lumens 145a-145d. The fluid lumens 145e-145k from the embodiment of FIG. 1E are not used as fluid lumens in this embodiment. They can be, for example, empty lumens, vacuum, filled with an insulative material, and/or filled with a radio-opaque material or any other material that may help visualize the thrombus removal system during therapy. The embodiment 1F includes the same four auxiliary reports as illustrated and described in the embodiment of FIG. 1E.

Section B-B of FIG. 1G illustrates another example of a portion of the thrombus removal system that is proximal to the funnel and irrigation manifold. Similar to the embodiments described above, the illustrated portion of the thrombus removal system can include a middle wall 170 disposed between the outer wall 140 and the inner wall 150. However, this embodiment includes four distinct fluid lumens 145a-145d formed by wall structures 165. As with the embodiment of FIG. 1C, the wall structures 165 can be formed by lamination between the outer and inner walls 140 and 150, or by a multi-lumen extrusion that forms a plurality of the wall structures. As shown, this embodiment can include a pair of auxiliary lumens 175a and 175b, which can be used, for example, for steering or for sensor connections as described above.

Section B-B of FIG. 1H is another similar embodiment in which the middle wall and outer wall can be used to form fluid lumens 145a and 145b. Auxiliary lumens 175a and 175b can be formed in the space between the middle wall and the inner wall. It should be understood that the middle wall can contact the outer wall to create independent fluid lumens 145a and 145b. However, in other embodiments, it should be understood that the middle wall may not contact the outer wall, which would facilitate a single annular fluid lumen, such as is shown by fluid lumen 145 in Section B-B of FIG. 1I. In another embodiment, as shown in Section B-B of FIG. 1J, the inner wall 150 and the outer wall 140 may not be concentric, which facilitates formation of an annular space and/or fluid lumen 145 that is thicker or wider on one side of the device relative to the other side. As shown in FIG. 1J, a distance between the exemplary outer wall 140 and inner wall at the top (e.g., 12 o'clock) portion of the device is larger than a distance between the outer wall and inner wall at the bottom (e.g., 6 o'clock) portion of the device.

Section C-C of FIG. 1K illustrates in plan view a portion of the thrombus removal system comprising an irrigation manifold 225. Section C-C depicts an outer wall 240, an inner wall 250, a fluid lumen 245, an aspiration lumen 255, and ports 230 for directing respective fluid streams 210.

Detail View 101 of FIG. 1L illustrates a section view in elevation of a portion of the irrigation manifold 25 that includes a plurality of ports 230 that are formed within an inner wall 250. In some embodiments, a thickness of one or more walls of the thrombus removal system may be varied along its axial length and/or its circumference. As shown in Detail View 101, inner wall 250 has a first thickness 265 in a region 250 that is proximal to the irrigation manifold 25, and a second thickness 270 in a region 235 that includes the ports 230. In some embodiments, the second thickness 270 is greater than the first thickness 265. The first thickness 265 can correspond to a general wall thickness of the inner wall 50 and/or of the outer wall 40, which can be from about 0.10 mm to about 0.60 mm, or any value within the aforementioned range. The second thickness 270 can be from about 0.20 mm to about 0.70 mm, from about 0.70 mm to about 0.90 mm, or from about 0.90 mm to about 1.20 mm. The second thickness 270 can be any value within the aforementioned range. The dimension of the second thickness 270 can be selected to provide a fluid path through the ports 230 that produces a generally laminar flow for a fluid stream that is directed therethrough, when the fluid delivery mechanism supplies fluid via the fluid lumen 245 at a typical operating pressure. Such operating pressure can be from about 10 psi to about 60 psi, from about 60 psi to about 100 psi, or from about 100 psi to about 150 psi. The operating pressure of the fluid delivery mechanism can be any value within the aforementioned range of values. In some embodiments, the fluid delivery mechanism is operated in a high pressure mode, having a pressure from about 150 psi to about 250 psi, from about 250 psi to about 350 psi, from about 350 psi to about 425 psi, or from about 425 psi to about 500 psi. The operating pressure of the fluid delivery mechanism in the high pressure mode can be any value within the aforementioned range of values.

The manifold is configured to increase a fluid pressure and/or flow rate of the fluid. When fluid is provided by the fluid delivery mechanism to the fluid lumen(s) at a first pressure and/or a first flow rate, the manifold is configured to increase the pressure of the fluid to a second pressure and/or is configured to increase the flow rate of the fluid to a second flow rate. The second pressure and/or second fluid rate can be higher than the first pressure and/or first flow rate. As a result, the manifold can be configured to increase the relatively low operating pressures and/or flow rates generated by the fluid delivery mechanism to the relatively high pressures and/or high flow rates generated by the ports/fluid streams.

In some embodiments, a profile (cross-sectional dimension) of a port 230 varies along its length (e.g., is non-cylindrical). A variation in the cross-sectional dimension of the port may alter and/or adjust a characteristic of fluid flow along the port 230. For example, a reduction in cross-sectional dimension may accelerate a flow of fluid through the port 230 (for a given volume of fluid). In some embodiments, a port 230 may be conical along its length (e.g., tapered), such that its smallest dimension is positioned at the distal end of the port 230, where distal is with respect to a direction of fluid flow.

In some embodiments, the port 230 is formed to direct the fluid flow along a selected path. FIGS. 2A-2E illustrate various embodiments of arrangements of ports 230 for directing respective fluid streams 210. In some embodiments, such as those shown in FIGS. 2A and 2B, at least two ports 230 are arranged to produce (e.g., respective) fluid streams 210 that intersect at an intersection region 237 of the thrombus removal system. An intersection region 237 can be a region of increased fluid momentum and/or energy transfer, which multiply with respect to individual fluid streams that are not directed to combine at the intersection. The increased fluid momentum and/or energy transfer at an intersection may advantageously fragment thrombus more efficiently and/or quickly. As described above, the fluid streams can be configured to accelerate and cause cavitation and/or other effects to further add to breaking up of the target clot. In some embodiments, an intersection region can be formed from at least 2, at least 3, at least 4, at least 5, at least 6, at least 7, at least 8, at least 9, or at least 10 fluid streams 210. An intersection region can be generally near a central axis 290 of the thrombus removal system (e.g., 237), or away from the central axis (e.g., 238 and 239 in the embodiment of FIG. 2D). In some embodiments, at least two intersection regions (e.g., 238 and 239) are formed. In some embodiments, one or more ports 230 are arranged to direct a fluid stream 210 along an oblique angle with respect to the central axis of the thrombus removal system. An operating pressure of the fluid delivery mechanism may be selected to approach a minimum targeted fluid velocity for a fluid stream 210 that is delivered from a port 230. The targeted fluid velocity for a fluid stream 210 can be about 5 meters/second (m/s), about 8 m/s, about 10 m/s, about 12 m/s, or about 15 m/s. Additionally, the targeted fluid velocities in some embodiments can be in the range above 15 m/s to up to 150 m/s. At these higher velocities (e.g. above 15 m/s, or alternatively above 20 m/s), the fluid streams may be configured to generate cavitation in a target thrombus or tissue. It has been found that with fluid exiting from the ports to these flow rates a cavitation effect can be created in the focal area of the intersecting or colliding fluid streams, or additionally at a boundary of one or more of the fluid streams. While the exact specifications may change based on the catheter size, in general, at least one of the fluid streams should be accelerated to such a high velocity to create cavitation as described in detail below. The targeted fluid velocity for fluid stream 210 can be any value within the range of aforementioned values. In some embodiments, at least two ports 230 are adapted to deliver respective fluid streams at different fluid velocities (i.e. speed and direction), for a given pressure of the fluid delivery mechanism. In some embodiments, at least two ports 230 are adapted to deliver respective fluid streams at the substantially the same fluid velocities, for a given pressure of the fluid delivery mechanism. In some embodiments, one port is adapted to deliver fluid at high velocity and the respective one or more other ports is adapted to deliver fluid at relatively lower velocities. Advantageously, an increased cross-sectional area of the fluid lumen 145 reduces a required operating pressure of the fluid delivery mechanism to achieve a targeted fluid velocity of the fluid streams.

In some embodiments, the fluid streams are configured to create angular momentum that is imparted to a thrombus. In some examples, angular momentum is imparted on the thrombus by application of a) at least one fluid stream 210 that is directed at an oblique angle from a port 230, and/or b) at least two fluid streams 210 that have different fluid velocities. For example, fluid streams that cross near each other but do not necessarily intersect may create a “swirl” or rotational energy on the clot material. Advantageously, angular momentum produced in a thrombus may impart a (e.g., centrifugal) force that assists in fragmentation and removal of the thrombus. Rotating of the clot may enhance delivery of the clot material to the jets. By example, with a large, amorphous clot the soft material may be easily aspirated or broken up by the fluid streams whereas tough fibrin may be positioned away from the fluid streams. Rotating or swirling of the clot moves the material around so the harder clot material is presented to the jets. The swirling may also further break up the clot as it is banged inside the funnel.

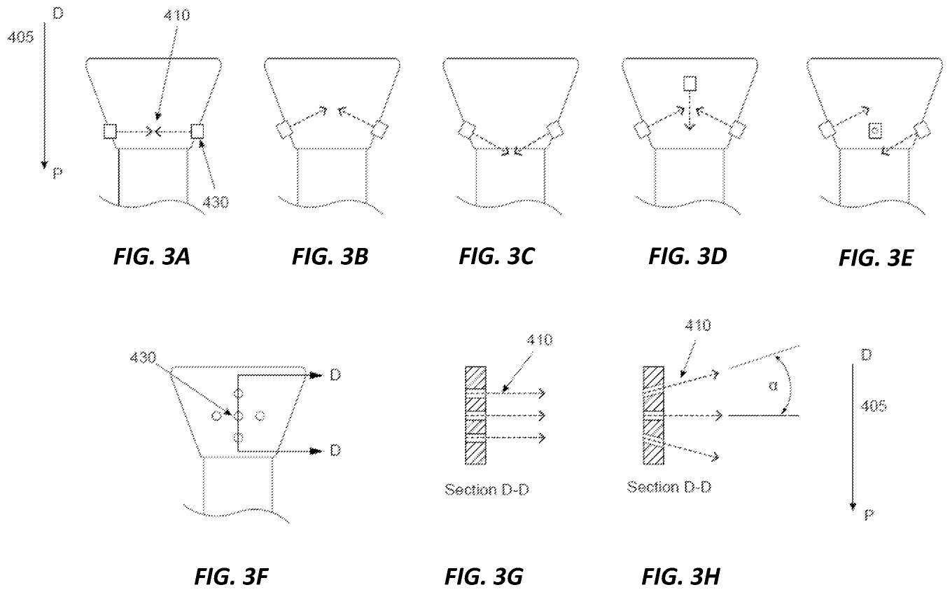

FIGS. 3A-3H depict various configurations of fluid streams 410 that are directed from respective ports 430. A fluid stream 410 can be directed along a path that is substantially orthogonal, proximal, and/or distal to the flow axis 405 (which is like to flow axis 305). In some embodiments, at least two fluid streams are directed in different directions with respect to the flow axis 405. In some embodiments, at least two fluid streams are directed in a same direction (e.g., proximally) with respect to the flow axis 405. In some embodiments, at least a first fluid stream is directed orthogonally, at least a second fluid stream is directed proximally, and at least a third fluid stream is directed distally with respect to the flow axis 405. An angle a may characterize an angle that a fluid stream 410 is directed with respect to an axis that is orthogonal to the flow axis 405 (e.g., as shown in section D-D of FIGS. 3G and 3H). An intersection region of fluid streams can be within an interior portion of the thrombus removal system, and/or exterior (e.g., distal) to the thrombus removal system. In some embodiments, a fluid stream that is directed by a port 430 in a nominal direction (e.g., distally) is deflected along an altered path (e.g., proximally) by (e.g., suction) pressure generated by the aspiration mechanism during operation.

### Cavitation Generation

The exemplary system includes fluidic jets configured in a particular manner to enhance removal of clot. The exemplary fluid streams or jets have been shown in bench studies to dramatically improve removal of clot through various mechanisms of action including, but not limited to, cavitation and water cutting. In contrast to conventional fluid mechanisms for thrombectomy, in some embodiments herein, fluid streams from respective ports are delivered at sufficient flow rates (and patterns) to create cavitation and/or other preferential effects to improve removal of clot. In certain examples, the cavitation effect is created by large pressure drops and deceleration at the focal point and/or intersection point of at least two fluid streams. The cavitation may provide a source of turbulent kinetic energy that can be used to mechanically fractionate and/or liquefy thrombi or other target tissue structures. When the fluid velocity is sufficiently high, the material accumulates impact energy, which can cause deformation and fragmentation. This also may modify the surface properties of the clot to allow the material to be penetrated to enable cavitation within the clot. Collision or interaction of the high-speed jets creates hydrodynamic cavitation whereby a pressure drop below the vapor pressure of the liquid creates bubbles which eventually collapse with great mechanical energy in the cavitation field, causing a kind of implosion in the clot material. Further, with multiple jets directed towards a focal point or sufficiently near respective streams, the closing speed of the fluid particles is significantly higher (up to double) that of a single jet stream. This also forces fluid and/or particles out from the space between the fluid jets at high speed. The speed of the fluid jets is sufficiently high to create a pressure drop below the vapor pressure such that the fluid vaporizes. When pressure rises again the bubble collapses, which causes the cavitation. It has been found that the power of the exemplary system and cavitation effect significantly exceeds conventional fluid jet(s) and mechanical tools like rotating screws. In some examples, the collapse of the bubbles may generate heat in or around the target tissue, which may further promote breaking up of the clot. In bench studies systems in accordance with various embodiments were able to remove certain clot material that simple aspiration or water jetting were not. In other studies, the exemplary systems were able to remove clot material in a fraction of the time of conventional systems.

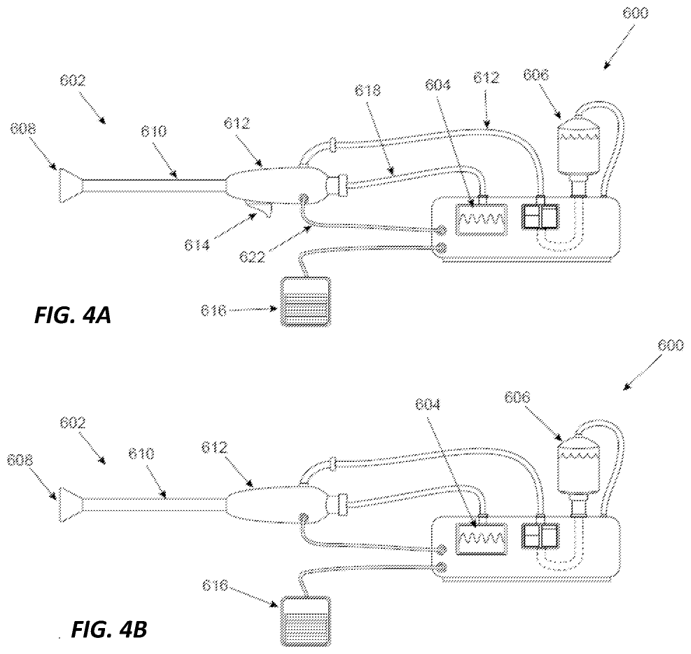

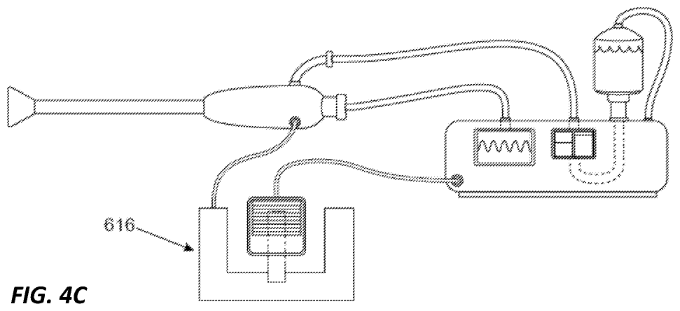

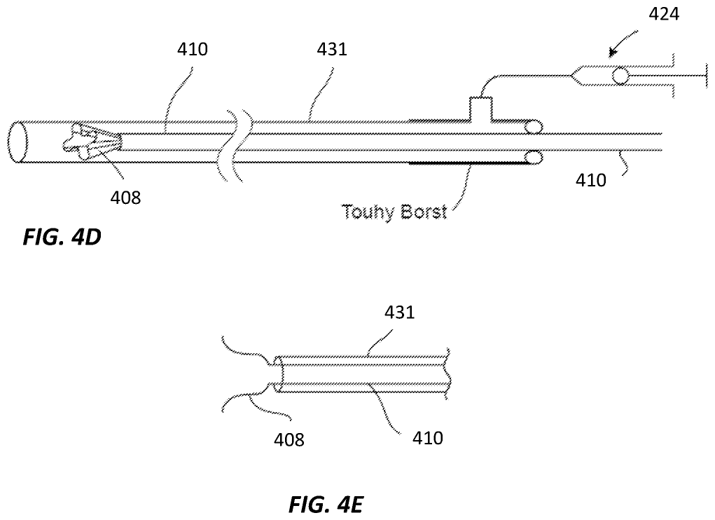

FIGS. 4A-4C illustrate various configurations of a thrombus removal system 600, including a thrombus removal device, 602, a vacuum source and cannister 604, and a fluid source 606. In some embodiments, the vacuum source and cannister and the fluid source are housed in a console unit that is detachably connected to the thrombus removal device. A fluid pump can be housed in the console, or alternatively, in the handle of the device. The console can include one or more CPUs, electronic controllers, or microcontrollers configured to control all functions of the system. The thrombus removal device 602 can include a funnel 608, a flexible shaft 610, a handle 612, and one or more controls 614 and 616. For example, in the embodiment shown in FIG. 4A, the device can include a finger switch or trigger 614 and a foot pedal or switch 616. These can be used to control aspiration and irrigation, respectively. Alternatively, as shown in the embodiment of FIG. 4B, the device can include only a foot switch 614, which can be used to control both functions, or in FIG. 4C, the device can include only an overpedal 616, also used to control both functions. It is also contemplated that an embodiment could include only a finger switch to control both aspiration and irrigation functions. As shown in FIG. 4A, the vacuum source can be coupled to the aspiration lumen of the device with a vacuum line 618. Any clots or other debris removed from a patient during therapy can be stored in the vacuum cannister 604. Similarly, the fluid source (e.g., a saline bag) can be coupled to the fluid lumens of the device with a fluid line 620.

Still referring to FIG. 4A, electronics line 622 can couple any electronics/sensors, etc. from the device to the console/controllers of the system. The system console including the CPUs/electronic controllers can be configured to monitor fluid and pressure levels and adjust them automatically or in real-time as needed. In some embodiments, the CPUs/electronic controllers are configured to control the vacuum and irrigation as well as electromechanically stop and start both systems in response to sensor data, such as pressure data, flow data, etc.

In FIG. 4D, a system assembly is shown including a funnel 408 and a flexible shaft 410 of a thrombus removal device inserted into a steerable introducer catheter 431. A hub assembly such as a Touhy Borst is shown which can provide access for a medical device into the steerable introducer catheter and include an injection port for fluidic connection to the contrast injector 424. In this embodiment, injection of contrast from the injector 424 into the hub assembly provides the contrast agent into the annular space between the introducer catheter 431 and the thrombus removal device (e.g., the shaft of the thrombus removal device).

FIG. 4E shows the funnel 408 of the thrombus removal device axially disposed out of a distal end of the introducer catheter 431. In this example, contrast delivered by the injector 424 into the annular space can still be delivered into the patient, even when the funnel is in a deployed configuration. In some examples, the funnel can disperse the contrast agent as it's delivered past the funnel from the annular space.

In some aspects, the hub assembly such as a Touhy Borst can provide access for the thrombectomy device into the lumen of the catheter sheath or introducer catheter. The hub assembly can further include an injection port for fluidic connection of a fluid or contrast source to the annular space between the catheter sheath and the thrombectomy catheter. In any of the embodiments described herein, the axial position of the external sheath (e.g., introducer catheter) can be locked or fixed relative to the axial position of the thrombectomy device or catheter. For example, the Touhy on the introducer hub can be used to lock the axial position of the catheter relative to the sheath.

As is described above, aspiration occurs down the central lumen of the device and is provided by a vacuum pump in the console. The vacuum pump can include a container that collects any thrombus or debris removed from the patient.

### Funnel With Adjustable Size and Geometry

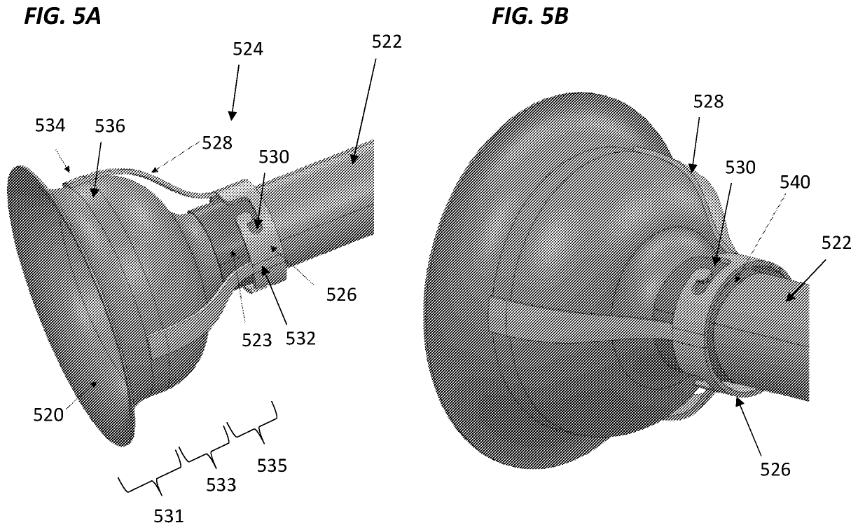

FIGS. 5A-5B illustrate an adjustable funnel 520 of a thrombus removal device that includes features configured to facilitate 1) changing a size/diameter of the funnel, and/or 2) changing a geometry of a deployed funnel. In some aspects, the size/diameter of the funnel or the geometry of the funnel can be changed/adjusted at various axial locations along the funnel. For example, a shape or size of a distal edge or portion of the funnel can be adjusted in some embodiments, while the shape or size of a proximal portion of the funnel can be adjusted in other embodiments. In some embodiments, a proximal or central portion of the funnel can be adjusted (e.g., shape/diameter). Variations are considered in which more than one axial position of the funnel can be adjusted in shape and/or size.

The adjustable funnel can include an adjustable frame that can be configured to enlarge or reduce a diameter of the funnel. For example, adjustability of the funnel diameter (e.g., a distal end of the funnel) allows for adjustment of the funnel size for capturing clots in smaller lumens (e.g., in the lobar pulmonary arteries) while still allowing for a larger funnel size for larger clots in larger lumens (e.g., in the pulmonary trunk). Additionally, in some embodiments the adjustable frame can also be configured to alter a geometry of the funnel. For example, the geometry of the funnel can be changed or adjusted between various shapes/geometries, including a circular, elliptical, skived, or clover shape. In some aspects, changing a shape of the funnel can comprise changing a cross-sectional shape of the funnel from generally circular to generally non-circular.

In some examples, the funnel shape can be changed to enhance clot movement into the jets (e.g., by increasing a diameter of the proximal portion of the funnel near the jets/aspiration lumen).

In some of the described embodiments herein, the funnel itself can include a funnel frame that provides structural support for compliant material of the funnel. The funnel frame can include, for example, a superelastic or nitinol frame that can be pre-biased to form an expanded state. In some examples, the funnel and funnel frame can be collapsed in a delivery sheath for delivery to a target tissue site, and when the funnel is advanced out of the sheath, the funnel frame can cause the funnel to assume an expanded configuration. In other embodiments, the funnel may comprise an entirely flexible or compliant material with no separate or distinct funnel frame. In this embodiment, the adjustable frame and/or frame ribs described below may provide all the structural stability for the frame when in the expanded state.

FIGS. 5A-5B show an adjustable funnel 520 can be disposed on or near a distal end of an elongate catheter shaft 522. The funnel can be collapsible for delivery and removal and expandable during therapy for capturing clots and/or engaging with a vessel wall. In some embodiments, the funnel can include a compliant material (e.g., a urethane material such as Chronoflex) to facilitate expansion and collapse of the funnel and also to avoid trauma to delicate vessel walls. In some examples, the funnel can be attached directly to the elongate catheter shaft. In other embodiments, as shown, a collar assembly 523 can be used to couple the funnel 520 to the elongate catheter shaft 522. While not explicitly illustrated in FIGS. 5A-5B, it should be understood that the device can include any number of jets or fluid ports disposed within the funnel or aspiration lumen, including any of the configurations as shown in FIGS. 1, 1A, 1L, 2A-2D, 3A-3H or otherwise described herein.

The adjustable funnel 520 can further include an adjustable frame 524 that can include a support frame collar 526, frame ribs 528, and one or more engagement features 530. While the frame 524 is shown as being external to the funnel (e.g., along an outer surface of the funnel), it should be understood that in some embodiments some or all of the frame can be incorporated into or positioned within the funnel. In the illustrated embodiment, the frame ribs are attached or connected to the adjustable funnel, but the support frame collar is not fixedly attached or connected to the catheter shaft or the optional collar assembly. As shown in FIG. 5B, a gap or clearance 540 can be provided between the support frame collar and the collar assembly 523 to allow for relative motion between the collar assembly and the support frame collar and/or elongate shaft (e.g., when the funnel is attached directly to the elongate catheter shaft and no collar assembly is used). It should therefore be understood that movement of the support frame collar in the axial direction can therefore cause a corresponding axial movement in the frame ribs. Since the frame ribs are attached to the funnel itself, movement of the support frame collar can cause movement of the funnel at the rib attachment points, causing expansion, collapse, or deformation of the funnel. It should also be understood that since the frame ribs are attached to the funnel, twisting or rotating the support frame collar relative to the elongate shaft can also result in changing a size or shape of the funnel.

The number of frame ribs chosen for the adjustable frame and the orientation of the frame ribs around the funnel determines the type(s) of deformations and/or size adjustments that can be applied to the funnel. In general, any number of frame ribs can be implemented, including one frame rib, two frame ribs, three frame ribs, four frame ribs, or more than four frame ribs. Additionally, the frame ribs can be placed symmetrically or asymmetrically around the perimeter of the funnel. Furthermore, in some embodiments, the attachment point of the ribs to the funnel can be spaced axially (e.g., one or more ribs can attach to the funnel more distally or proximally than one or more other ribs). Details on some of the deformations achievable with various numbers of frame ribs are described below.

In embodiments described herein where frame ribs control the shape and/or size of the funnel, a number of advantages are provided over funnels that achieve and maintain their shape with integrated frames. For example, various shapes and/or diameters of the funnel are possible that would not be achievable solely with a rigid/integrated frame within the funnel. Furthermore, the frame ribs facilitate various funnel configurations that, if implemented with a rigid frame in the funnel, would be impossible to re-sheath the funnel after deployment due to large sheathing forces (e.g., an enlarged proximal funnel diameter). Similarly, with rigid/integrated frames, a balance must be achieved between the stiffness of the expanded frame and the force required to re-sheath the funnel. However, in embodiments herein with the frame ribs, this compromise can be decoupled. When the frame ribs are locked in place (e.g., with the catheter sheath), the frame ribs can make the funnel very stiff and resistant to prolapse. However, when the sheath and frame ribs are unlocked or decoupled, the frame can be very soft and after very little resistance to sheathing, regardless of geometry.

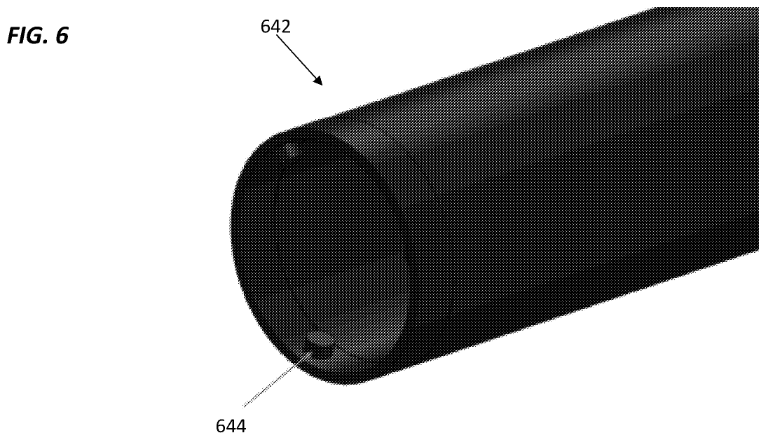

Furthermore, in some examples the frame ribs can be used to control the shape of the funnel, which may eliminate need to shape set the funnel to a final desired expanded shape. In some embodiments, the funnel can be manufactured in a desired geometry or in a partially expanded geometry. This could add a procedural benefit and make priming the device easier, as it would no longer require sheathing of a large sized funnel into an introducer prior to priming. Additionally, if the funnel is not shaped and instead uses its tubular as-cut configuration, the funnel will have a large, stored energy when expanded and locked at its given diameter with the frame ribs and sheath. In some aspects, the system can be configured to unlock or decouple the frame ribs from the sheath automatically when the system detects that a clot is cleared, allowing the funnel to close very quickly due to the stored strain energy of the shape memory material and act as a pinch valve at the distal end of the thrombectomy device. The one or more engagement features of 530 of the adjustable frame 524 are configured to engage with one or more corresponding engagement features of a separate catheter sheath (as shown in FIG. 6) that is positioned over the elongate catheter shaft.

In some embodiments, the frame ribs can be connected or attached to the support frame collar 526 at one or more attachment points 532. This can include, for example, welded attachment, riveted attachment, adhesive attachment, or a fixed attachment with screws or other features as known in the art. Alternatively, the ribs and support frame collar can be integral to another. In one embodiment, a distal portion of the frame ribs 528 can be attached directly to the funnel 520 itself at one or more attachment points 534. In other embodiments, the frame ribs 528 can be attached or connected to an optional peripheral frame structure 536 at one or more attachment points 534. The peripheral frame structure can be external to the funnel compliant material, or in other embodiments, the peripheral frame structure can be embedded in or sandwiched between one or more layers of the compliant material. While the embodiment of FIG. 5A shows the frame ribs external to the compliant material of the funnel, in other embodiments the frame ribs can be embedded in or sandwiched between layers of the compliant material.

The frame ribs 528 of the adjustable frame 524 can be connected at various axial positions along funnel. For example, the frame ribs can be connected to a central portion 533 of the funnel, as shown in FIG. 5A. Alternatively, the frame ribs can be connected or attached to a distal portion 531 of the funnel, or alternatively a proximal portion 535 of the funnel (e.g., near the jets and/or aspiration lumen of the device). In some embodiments, one or more frame ribs can be connected to one or more axial positions along the funnel (e.g., at least one frame rib connected to a first axial portion of the funnel and at least one frame rib connect to a different axial portion of the funnel).

FIG. 6 is a figure of the aforementioned catheter sheath 642 which is configured to reside over the elongate catheter shaft 522 of the thrombus removal device described above.

In some examples, an inner diameter (ID) of the catheter sheath 642 is larger than the outer diameter (OD) of the elongate catheter shaft 522 of the thrombus removal device. In some embodiments, the catheter sheath 642 can be an introducer sheath that is used to deliver the thrombus removal device to a target location within a patient's anatomy (e.g., in the pulmonary artery). In use, the thrombus removal device can be loaded into the catheter sheath, including the funnel which can assume a collapsed configuration within the catheter sheath for delivery to a target thrombus location.

As shown in FIG. 6, the catheter sheath 642 can include one or more engagement features 644 configured to interface with, engage, or key into the one or more engagement features 530 of the adjustable frame 524 described in FIGS. 5A-5B. While the engagement features 644 are shown as cylindrical tabs or posts, it should be understood that the engagement features are not limited to that specific shape or design. For example, the sheath 642 can comprise cutouts or slots configured to correspond with posts or tabs on the adjustable frame. Furthermore, in some embodiments, the sheath can be configured to manipulate or engage with the adjustable frame simply with axial movement (e.g., moving the sheath distally over the adjustable frame) to manipulate a size and/or shape of the funnel.

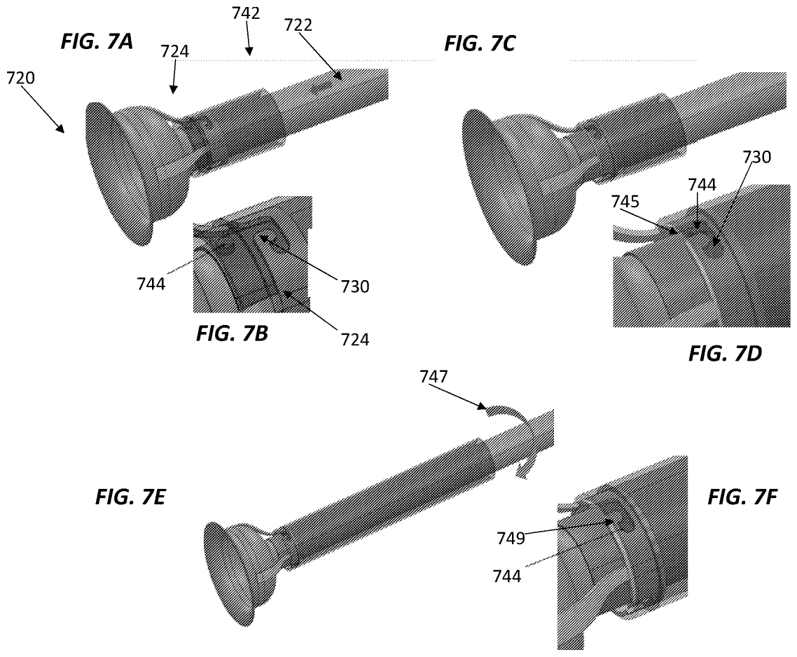

FIGS. 7A-7F illustrate techniques for manipulating the size/diameter and/or shape of the funnel of the thrombus removal device(s) described herein. In general, the engagement features of the catheter sheath can be engaged, locked, or keyed to the engagement features of the adjustable frame. When this connection is made, then the axial position of the support frame collar and frame ribs can be moved relative to the funnel/collar/elongate catheter shaft to adjust or change a size or shape of the funnel.

FIG. 7A shows a thrombus removal device with some or all of the features described above, including an elongate catheter shaft 722, a funnel 720, an adjustable frame 724 including a support frame collar 726 and frame ribs 728, and a catheter sheath 742 adapted to slide over the elongate catheter shaft. In FIG. 7A, the thrombus removal device is shown advanced axially through the sheath (e.g., in a distal direction) such that the funnel of the thrombus removal device is in a deployed configuration.

Referring to FIG. 7B, it should be noted that at this stage, the engagement feature 744 of the catheter sheath (e.g., the positive or male engagement feature previously described in FIG. 6) is positioned distally to the corresponding engagement feature 730 of the support frame collar (e.g., the engagement feature(s) 30 of FIGS. 5A-5B). To engage the engagement feature of the sheath with the engagement feature of the collar, the sheath is advanced distally beyond the collar and then pulled back proximally to engage. Other embodiments may allow for engagement between the sheath and collar my simply moving the sheath distally until contact is made with the collar to engage the corresponding engagement features.

In FIGS. 7C-7F, the engagement feature(s) 744 of the catheter sheath can be connected to, locked into, or engaged with the engagement feature(s) 730 of the support frame collar of the thrombus removal device. In this specific embodiment, first the engagement feature(s) of the catheter sheath can enter a keyway 745 of the support frame collar engagement mechanism(s), as shown in FIGS. 7C-7D. Next, in FIG. 7E, the catheter sheath can be rotated relative to the elongate shaft of the thrombus removal device, or vis versa (e.g., along arrow 747). As shown in FIG. 7F, this rotation causes the engagement feature(s) of the catheter sheath to lift up a tab 749 of the engagement feature(s) on the support frame collar to lock the catheter shaft in place with the adjustable frame.

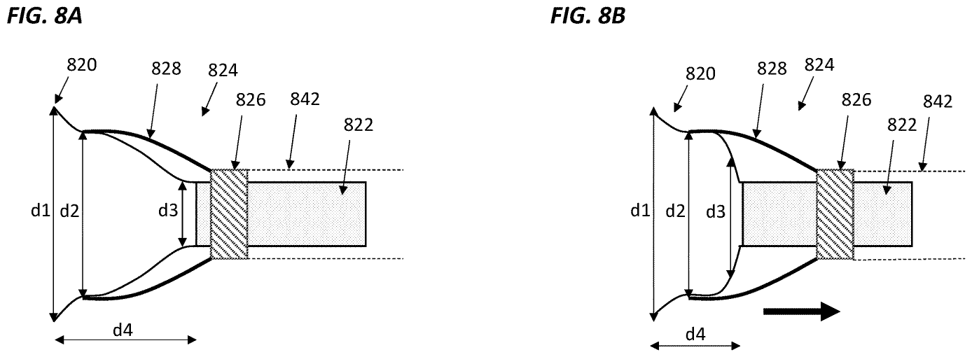

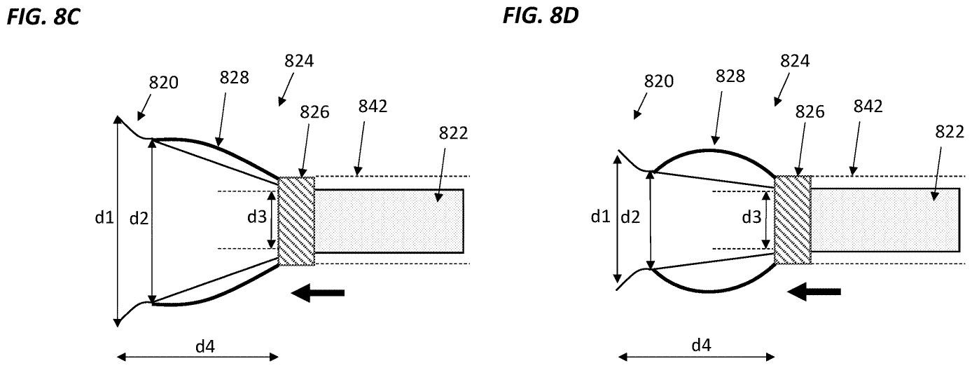

FIGS. 8A-8D illustrate additional embodiments of a thrombus removal system that can include any or all of the features described above, including elongate catheter shaft 822, funnel 820, adjustable frame 824, frame collar 826, frame ribs 828, and catheter sheath 842. While not explicitly illustrated in FIGS. 8A-8D, it should be understood that the device can include any number of jets or fluid ports disposed within the funnel or aspiration lumen, including any of the configurations as shown in FIGS. 1, 1A, 1L, 2A-2D, 3A-3H or otherwise described herein.

FIGS. 8A-8D illustrate side views of the manipulating the axial position of the adjustable frame to change the size or diameter of the funnel of the thrombus removal device. In some embodiments, the size or diameter of the funnel can be changed or adjusted in specific locations of the funnel. For example, the adjustable frame may be configured to only adjust the size or diameter of a base or proximal portion of the funnel. Alternatively, the adjustable frame may be configured to adjust a size or diameter of one or more of a distal portion of the funnel, a central portion of the funnel, and/or the base or proximal portion of the funnel.

Referring to FIG. 8A, the adjustable frame 824 of the thrombus removal device can be in an at-rest or non-actuated position relative to the elongate catheter shaft of the thrombus removal device. This can correspond, for example, to the position of the adjustable frame shown in FIGS. 5A-5B and 7A (e.g., before the engagement members of the sheath have been locked or connected with the engagement features of the support frame collar). As shown in this configuration, the funnel can have a distal portion diameter d1, central portion diameter d2 (corresponding to the attachment points of the frame ribs to the funnel), and a base or proximal portion diameter d3. Additionally, an axial length of the funnel is designated in FIG. 8A as distance d4. This can be, for example, a resting or shape-set configuration of the funnel, since the shape and size of the funnel is defined by any frame structures within the funnel itself (e.g., a superelastic or nitinol frame within the compliant material of the funnel).

FIG. 8B depicts an embodiment in which the catheter sheath 842 is engaged with the support frame collar 826 of the adjustable frame 824, and the adjustable frame 824 has been moved axially in the proximal direction with respect to the elongate catheter shaft 822. Moving the adjustable frame in the proximal direction relative to the elongate catheter shaft (or alternatively, moving the elongate catheter shaft distally with respect to the adjustable frame) can change a shape and/or diameter of the funnel as shown. Specifically, this movement can cause the base or proximal portion of the frame to expand in diameter. It can be seen in FIG. 8B how the base or proximal portion of the funnel can bow or move outwards towards the frame ribs 828 as the entire funnel is pulled proximally towards the elongate catheter shaft. While distal portion diameter d1 and central portion diameter d2 (at the attachment points of the frame ribs) generally remain the same as in FIG. 8A, the diameter of the base or proximal portion can be increased with this axial movement of the adjustable frame. As described previously, jets or fluid ports can be disposed at a base of the funnel near the aspiration lumen. The ability to widen the base or proximal portion of the funnel, as shown in FIG. 8B, can make it easier for clots within the funnel to reach the jets or fluid ports, and therefore make it easier for the thrombus removal device to macerate and remove the clots from the patient. It should be noted that moving the adjustable frame proximally relative to the funnel also causes the axial length d4 of the funnel to shrink or reduce compared to the at rest version of the funnel shown in FIG. 8A, thereby facilitating pulling the clot into the plane of the jets of the thrombus removal device.

FIGS. 8C-8D show another embodiment in which the catheter sheath 842 is engaged with the support frame collar 826 of the adjustable frame 824, and the adjustable frame 824 has been moved axially in the distal direction with respect to the elongate catheter shaft 822. Moving the adjustable frame in the distal direction relative to the elongate catheter shaft (or alternatively, moving the elongate catheter shaft proximally with respect to the adjustable frame) can change a shape and/or diameter of the funnel as shown. In both FIGS. 8C and 8D, this movement can cause the base or proximal portion of the frame to reduce in diameter. It can be seen in FIGS. 8C-8D how the funnel is stretched out by the distal movement of the frame ribs 828, causing the base or proximal diameter d3 to reduce relative to the at rest configuration of FIG. 8A. It should be noted that in this embodiment, the support frame collar 826 is advanced distally past the junction of the funnel and the aspiration lumen, so d3 is illustrated with dashed lines to indicate the true diameter at the base. In the embodiment of FIG. 8C distal portion diameter d1 and central portion diameter d2 (at the attachment points of the frame ribs) generally remain the same as in FIG. 8A. However, it should be appreciated that in other embodiments the flexibility of the frame ribs 828 can be selected to cause them to bow or flex inwards when the distal force is applied by the frame ribs to the funnel. In this example, shown in FIG. 8D, a distal end of the frame ribs 828 bow or flex inwards, causing the distal diameter d1, the central diameter d2, and the base or proximal diameter d3 to all be reduced relative to the at-rest state shown in FIG. 8A.

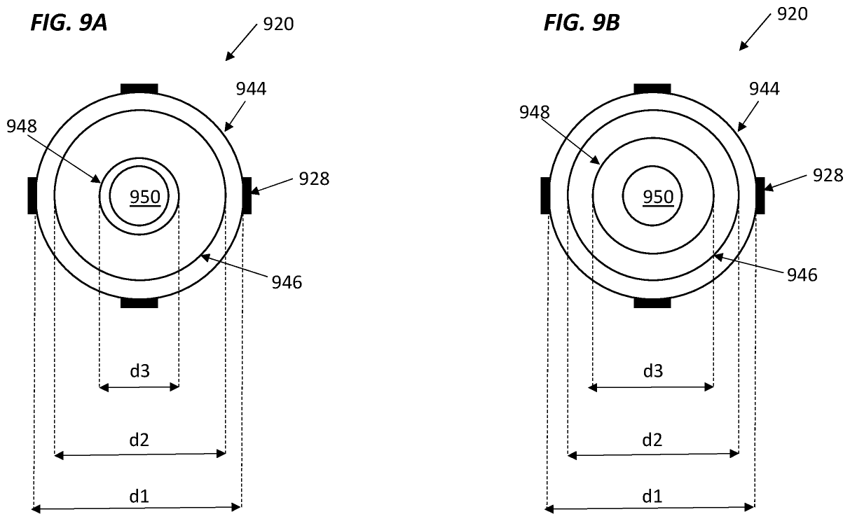

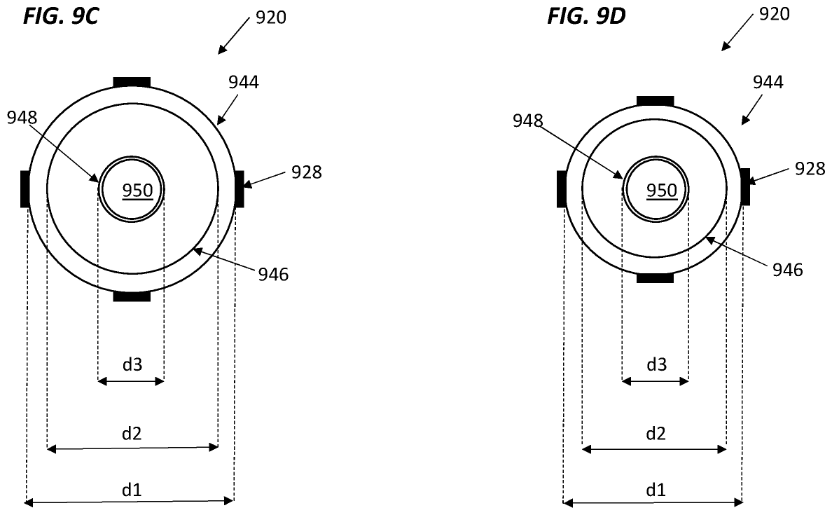

FIGS. 9A-9D are top-down views of the thrombus removal device corresponding to side-views of FIGS. 8A-8D, respectively. FIGS. 9A-9D illustrate the distal portion of the funnel 944, a central portion of the funnel 946, and a proximal or base portion of the funnel 948 (which can correspond, for example, to distal portion 531, central portion 533, and proximal portion 535 in FIG. 5A). Additionally, the aspiration lumen 950 of the elongate catheter shaft is shown. While not explicitly illustrated in FIGS. 9A-9D, it should be understood that the device can include any number of jets or fluid ports disposed within the funnel or aspiration lumen, including any of the configurations as shown in FIGS. 1, 1A, 1L, 2A-2D, 3A-3H or otherwise described herein. In these embodiments, four frame ribs are shown in symmetrical configuration, but it should be understood that any number and orientation of frame ribs can be provided. Generally, the more symmetrical and/or higher number of frame ribs provided allows for more symmetrical shaping of the funnel itself (e.g., an at rest circular configuration can be maintained during shaping with more frame ribs and/or more symmetrical frame rib placement).

FIG. 9A shows a top-down view of an at-rest configuration of a funnel 920 with frame ribs 928, the funnel having distal portion 944 with a diameter d1, a central portion 946 with a diameter d2, and a base or proximal portion 948 with a diameter d3. This top-down view can correspond to the side-view shown in FIG. 8A.

FIG. 9B shows a top-down view corresponding to the side-view of FIG. 8B. In this embodiment, the adjustable frame has been moved proximally relative to the elongate catheter shaft to cause the base or proximal portion of the funnel to expand in diameter. It can be seen that diameter d3 of the base portion is larger than diameter d3 of the at-rest configuration of FIG. 9A.

FIGS. 9C-9D are top-down views corresponding to the side-views of FIGS. 8C-8D, respectively. In this embodiment, the adjustable frame has been moved distally relative to the elongate catheter shaft to cause the base or proximal portion of the funnel to narrow or reduce in diameter. In the embodiment of FIG. 9C, the frame ribs are relatively stiff compared to the stiffness of the funnel, so distally advancing the ribs causes the base portion of the funnel to decrease in diameter but the distal and central portions of the funnel maintain approximately the same diameter as the at-rest configuration. However, as described above, in the embodiment of FIG. 9D, the frame ribs can have some compliance or flexibility that causes them to bow or flex inwards into the funnel when the adjustable frame is extended in the distal direction. This can cause all aspects of the funnel, including distal portion 944, central portion 946, and/or proximal or base portion 948 to narrow or decrease in diameter relative to the at-rest configuration of FIG. 9A.

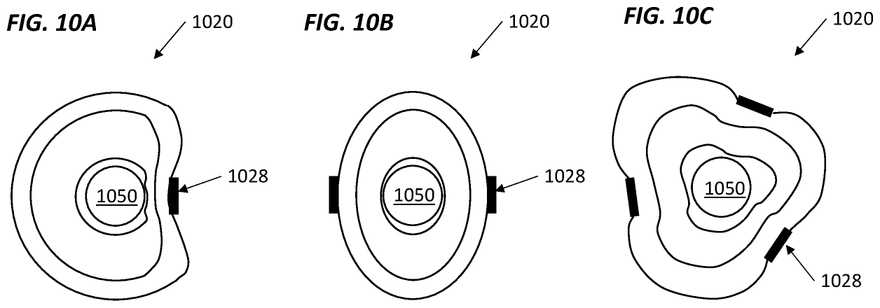

FIGS. 10A-10C illustrate embodiments where different numbers of frame ribs 1028 and/or asymmetric placement of frame ribs around the perimeter of the funnel 1020 and aspiration lumen 1050 can be used to control a shape of the funnel when the adjustable frame is actuated or moved relative to the elongate catheter shaft. Referring to FIG. 10A, a single rib can apply a skive shape or cross-section to the funnel when the adjustable frame is actuated. As shown, this skive shape can apply to some or all of the funnel sections, including the distal, central, and proximal or base funnel sections. In some embodiments, the skive shape can apply an indentation or reduced funnel diameter in the location of the frame rib.

In FIG. 10B, two frame ribs on opposite or symmetric opposing sides of the funnel can apply an oval or oblong shape to the funnel when actuated, as shown. Once again, this oval shape can apply to some or all of the funnel sections, including the distal, central, and proximal or base funnel sections. The axial location of the frame rib attachment to the funnel can determine which section or portion of the funnel is affected by manipulation of the frame ribs. For example, attachment of the frame ribs to a distal portion may apply an oval or oblong shape to the distal portion of the funnel, and potentially to a less extent the central portion of the funnel, while it may also not affect the shape of the proximal portion. Other factors can be tuned in the system to further control how the funnel responds to frame rib manipulation, including for example the stiffness/thickness of the funnel and any additional funnel frame materials or supports.

FIG. 10C illustrates an example where three frame ribs are applied symmetrically around the funnel to apply a clover-like shape to the funnel. As described above, the axial location of the frame ribs on the funnel determines which sections of the funnel are more affected by the shaping or adjustment of the funnel shape.

While the embodiments of FIGS. 10A-10C only show a top-down view, it should be understood that applying directional forces to only the connection points between the frame ribs and the funnel may also cause deformations or shape changes in the axial or side-profile directions of the funnel. These deformations or shapes can then be used to advantageously capture clot and/or direct clots into the funnel and towards the jets and aspiration lumen of the thrombus removal device.

### Other Techniques for Manipulating the Funnel Shape And/or Diameter

The embodiments discussed above generally describe an adjustable frame of a funnel that can be manipulated (e.g., with a catheter sheath) to adjust or change a shape and/or diameter(s) of the funnel. However, it should be understood that other techniques for manipulating the adjustable frame (e.g., the frame ribs) are contemplated within the scope of this disclosure. For example, any of the adjustable frames and/or frame ribs described herein can be directly coupled to one or more shafts, cables, pull wires, or similar structures to allow for manipulation of the adjustable frame and/or frame ribs from a proximal portion of the device. The one or more shafts, cables, pull wires, fluid columns, etc. may run along the length of the elongate catheter shaft from the adjustable frame to a handle or user interface of the device. In some embodiments, these coupling structures are mechanically controlled by a user (e.g., by interacting with a button, lever, knob, wheel, etc.) In other embodiments, these coupling structures are further coupled to an automated actuation device such as a motor, pneumatic source, vacuum or fluid source, etc.

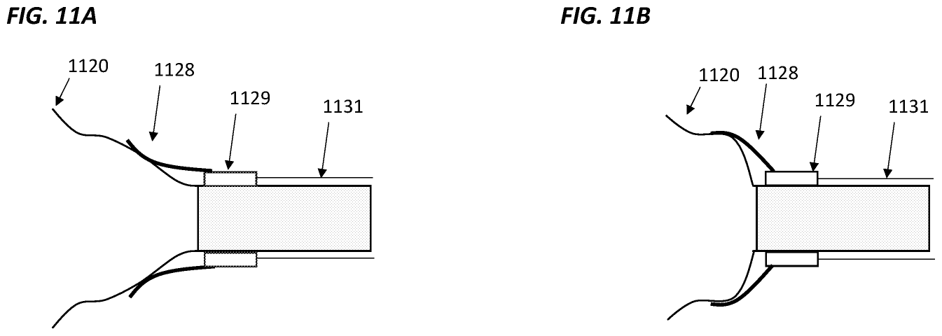

In some examples, the adjustable frame and/or frame ribs can be shape set (e.g., out of a shape memory material such as Nitinol) so when heated/cooled across a transition temperature, the shape of the frame/ribs will change to modify the funnel. For example, FIG. 11A shows a funnel 1120 of a thrombectomy device with a plurality of frame ribs 1128. The frame ribs can be coupled to, attached to, or integrated with contact points 1129, which can be coupled to, for example, one or more leads or wires 1131. As shown in FIG. 11B, when the ribs are heated (such as by applying electrical current to connection points 1129 with leads 1131) the frame ribs are configured to bend back on itself slightly (e.g., shifting from a concave orientation to a convex orientation or vis versa). In this illustrated embodiment, a concave structure is formed. This configuration better enables capturing clot by increasing or expanding a diameter of the funnel. As described above, the attachment point of the frame ribs to the funnel can determine which portion of the funnel is shaped or increased/decreased in diameter. In some embodiments, it is desirable to increase a diameter of a proximal portion of the funnel to assist with pulling clots into a jet plane and/or aspiration lumen of the device.

Various other configurations may be employed as would be understood by one of skill from the description herein. For example, the catheter system may include inner and outer frame elements. The inner structure may be formed as a frame for the funnel. The outer structure may be formed as an integrated rib structure and funnel frame. Other nested frame designs are contemplated. In various embodiments, the outside frame with ribs can be attached (e.g. riveted) to a section of the funnel frame. In the unactuated state, the ribs may be relatively straight and flush with the funnel walls as shown in FIG. 11A. With heat applied in the actuated state, the ribs change to a more concave geometry which would pull back on the funnel, foreshorten its height, and increase the diameter of the distal tip and/or proximal portion of the funnel as shown in FIG. 11B. This may be used to turn funnel into a wall scraper for adhered clot cases depending on the application.

While the embodiments herein have been described as being intended to remove thrombi from a patient's vasculature, other applications of this technology are provided. For example, the devices described herein can be used for breaking up and removing hardened stool from the digestive tract of a patient, such as from the intestines or colon of a patient. In one embodiment, the device can be inserted into a colon or intestine of the patient (such as through the anus) and advanced to the site of hardened stool. Next, the aspiration system can be activated to engage the hardened stool with an engagement member (e.g., funnel) of the device. Finally, the jets or irrigation can be activated to break off pieces of the hardened stool and aspirate them into the system. Any of the techniques described above with respect to controlling the system or removing clots can be applied to the removal of hardened stool.

As one of skill in the art will appreciate from the disclosure herein, various components of the thrombus removal systems described above can be omitted without deviating from the scope of the present technology. As discussed previously, for example, the present technology can be used and/or modified to remove other types of emboli that may occlude a blood vessel, such as fat, tissue, or a foreign substance. Further, although some embodiments herein are described in the context of thrombus removal from a pulmonary artery, the disclosed technology may be applied to removal of thrombi and/or emboli from other portions of the vasculature (e.g., in neurovascular, coronary, or peripheral applications). Likewise, additional components not explicitly described above may be added to the thrombus removal systems without deviating from the scope of the present technology. Accordingly, the systems described herein are not limited to those configurations expressly identified, but rather encompasses variations and alterations of the described systems.

### Conclusion

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A thrombus removal device, comprising:
an elongate shaft;
at least one aspiration lumen in the elongate shaft;
a funnel disposed at or near a distal end of the elongate shaft; and
an adjustable frame attached to the funnel and axially adjustable relative to the elongate shaft, wherein axial movement of the adjustable frame is configured to adjust a diameter of the funnel and/or a shape of the funnel.

2. The thrombus removal device of claim 1, wherein axial movement of the adjustable frame is configured to reduce a diameter of the funnel.

3. The thrombus removal device of claim 1, wherein axial movement of the adjustable frame is configured to reduce a diameter of a base portion of the funnel.

4. The thrombus removal device of claim 1, wherein axial movement of the adjustable frame is configured to reduce a diameter of a central portion of the funnel.

5. The thrombus removal device of claim 1, wherein axial movement of the adjustable frame is configured to reduce a diameter of a distal portion of the funnel.

6. The thrombus removal device of claim 1, wherein axial movement of the adjustable frame is configured to increase a diameter of the funnel.

7. The thrombus removal device of claim 1, wherein axial movement of the adjustable frame is configured to increase a diameter of a base portion of the funnel.

8. The thrombus removal device of claim 1, wherein axial movement of the adjustable frame is configured to increase a diameter of a central portion of the funnel.

9. The thrombus removal device of claim 1, wherein axial movement of the adjustable frame is configured to increase a diameter of a distal portion of the funnel.

10. The thrombus removal device of claim 1, wherein the adjustable frame comprises:
a support frame collar assembly disposed around the elongate shaft and configured to move axially relative to the elongate shaft; and
one or more frame ribs coupled to the support frame collar assembly and to the funnel.

11. The thrombus removal device of claim 1, further comprising a gap between the support frame collar and the elongate shaft.

12. The thrombus removal device of claim 1, further comprising a catheter sheath disposed over the elongate shaft, wherein the adjustable frame comprises one or more engagement features configured to couple with one or more engagement features of the catheter sheath.

13. The thrombus removal device of claim 12, wherein axial movement of the catheter sheath relative to the elongate shaft is configured to cause a corresponding axial movement of the adjustment frame when the catheter shaft is coupled to the adjustment frame.

14. The thrombus removal device of claim 1, wherein axial movement of the adjustable frame is configured to cause the funnel to assume an oval shape.

15. The thrombus removal device of claim 1, wherein axial movement of the adjustable frame is configured to cause the funnel to assume a skived shape.

16. The thrombus removal device of claim 1, wherein axial movement of the adjustable frame is configured to cause the funnel to assume a clover shape.

17. The thrombus removal device of claim 1, wherein the funnel comprises a circular cross section shape when at-rest, and wherein axial movement of the adjustable frame causes the funnel to assume a non-circular cross-section shape.

18. The thrombus removal device of claim 1, wherein the funnel comprises a compliant material and does not include a separate funnel frame for structure.

19. The thrombus removal device of claim 1, further comprising one or more coupling structures attached to the adjustable frame and extending proximally towards a handle of the device.

20. The thrombus removal device of claim 19, wherein the one or more coupling structures comprise pull wires.

21. The thrombus removal device of claim 19, wherein manipulation of the one or more coupling structures causes axial movement of the adjustable frame.

22-55. (canceled)
